(19) United States
(12) Patent Application Publication
(10) Pub. No.: US 2005/0188697 A1
(43) Pub. Date: Sep. 1, 2005

(54) Fluorinated ketone and fluorinated ethers as working fluids for thermal energy conversion

(75) Inventors: Gary J. Zyhowski, Lancaster, NY (US); Rajiv R. Singh, Getzville, NY (US)

Correspondence Address: Colleen D. Szuch, Esq.;Honeywell, Law Department, AB2, P.O. Box 2245, Morristown, NJ, 07962-2245, US

(73) Assignee: HONEYWELL CORPORATION

(21) Appl. No.: 10/790,303

(22) Filed: Mar. 1, 2004

(51) Int. Cl. A61K 31/44; F01K 25/00

(52) U.S. Cl. 60/671

(57) ABSTRACT

Polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl (perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof meet the requirement for not adversely affect atmospheric chemistry and would be a negligible contributor to ozone depletion and to green-house global warming in comparison to the fully halogenated hydrocarbons and are suitable for use as working fluids for u se i n thermal energy conversion systems such as an organic Rankine cycle system.

### FIELD OF THE INVENTION

This invention relates to the use of fluorinated ethers and fluorinated ketone as working fluids for thermal energy conversion. More particularly, this invention relates relates to the use of fluorinated ethers and fluorinated ketone as working fluids for conversion of thermal energy into mechanical energy in Rankine cycle systems.

### BACKGROUND OF THE INVENTION

Water, usually in the form of steam, is by far the most commonly employed working fluid used to help convert thermal energy into mechanical energy, This is due to its wide availability, low cost, thermal stability, non toxic nature and wide potential working range, to name a few of its virtues. However, other fluids have been utilized in certain applications, such as ammonia, in devices called Ocean Thermal Energy Conversion (OTEC) systems. In some instances, other fluids, such as CFC-113, have been utilized to recover energy from waste heat, such as exhausts from gas turbines. Another possibility is to employ two working fluids, such as water for the high temperature/pressure first part and a more volatile fluid in a cooler second stage. These hybrid poweer systems (also commonly refered to as binary power systems) can be more efficient than when only water and/or steam is employed.

In the quest to have a secure and reliable power source, data centers, military installations, government buildings, and hotels, for example, have come to use distributed power generation systems. To avoid loss of service that can occur with loss of grid power, including extensive cascading power outages that can occur when equipment designed to prevent such an occurrence fails, the use of distributed power generation is likely to grow. Typically, an on-site prime mover such as a gas microturbine would drive an electric generator and manufacture electricity for on-site use. The system would be connected to the grid or could run independent of the grid in some circumstances. Similarly, internal combustion engines capable of running on different fuel sources are used in distributed power generation. Fuel cells are also being commercialized for distributed power generation. Waste heat from these sources as well as waste heat from industrial operations, landfill flares, and heat from solar and geothermal sources can be used for thermal energy conversion. The typical approach is to use an organic working fluid in a Rankine cycle (instead of water) for cases where low- to medium-grade thermal energy is available. This is largely due to the high volumes, that is, large equipment sizes that would need to be accommodated if water were used as the working fluid at these low temperatures.

From thermodynamic considerations, the greater the difference between source and sink temperatures, the higher the organic Rankine cycle efficiency. It follows that organic Rankine cycle system efficiency is influenced by the ability to match a working fluid to the source temperature. The closer the evaporating temperature of the working fluid is to the source temperature, the higher the efficiency will be. Therefore, the higher the working fluid critical temperature, the higher the efficiency that can be attained. However, there are also practical considerations for thermal stability, flammability, and materials compatibility that bear on the selection of a working fluid. For instance, to access high temperature waste heat sources, toluene is often used as a working fluid. However, toluene is flammable and has toxicological concerns. In the temperature range of 175° F. to 500° F. (79° C. to 260° C.), non-flammable fluids such as HCFC-123 (1,1-dichloro-2,2,2-trifluoroethane and HFC-245fa (1,1,1,3,3-pentafluoropropane) are used. However, HCFC-123 has a relatively low permissible exposure level and is known to form toxic HCFC-133a at temperatures below 300° F. To avoid thermal decomposition, HCFC-123 may be limited to an evaporating temperature of 200° F.-250° F. (93° C.-121° C.). This limits the cycle efficiency and work output. In the case of HFC-245fa, the critical temperature is lower than optimum. Unless more robust equipment is used to employ a trans-critical cycle, the HFC-245fa organic Rankine cycle is held below the 309° F. (154° C.) critical temperature. To increase the useful work output and/or efficiency of an organic Rankine cycle beyond the limitations noted above for HCFC-123 and HFC-245fa, it becomes necessary to find working fluids with higher critical temperatures so that available source temperatures such as gas turbine and internal combustion engine exhaust can be approached more closely.

Certain members of a class of chemicals known as HFCs (hydrofluorocarbons) have recently been investigateded as substitutes for compounds known as CFC's (chlorofluorocarbons) and HCFC's (hydrochlorofluorocarbons), both which have been shown to be deleterious to the planet's atmospheric ozone layer. The initial thrust of the HFC development was to produce nonflammable, non-toxic, stable compounds that could be used in air conditioning/heat pump/insulating applications. However, few of these HFCs have boiling points much above room temperature. As was mentioned above, working fluids with critical temperatures higher than, for example, HFC-245fa, are desirable. Since boiling point parallels critical temperature, it follows that fluids with higher boiling points than HFC-245fa are desired.

A feature of certain hydrofluoropropanes, including HFC-245fa as compared to fluoroethanes and fluoromethanes, is a higher heat capacity due, in part, to an increase in the vibrational component contribution. Essentially, the longer chain length contributes to the freedom to vibrate; noting, of course, that the constituents and their relative location on the molecule also influence the vibrational component. Higher heat capacity contributes to higher cycle efficiency due to an increased work extraction component and also an increase in overall system efficiency due to improved thermal energy utilization (higher percentage of the available thermal energy is accessed in sensible heating). Moreover, the smaller the ratio of latent heat of vaporization to heat capacity, the less likely there will be any significant pinch point effects in heat exchanger performance. Hence, in comparison to HFC-245fa and HCFC-123, working fluids that posess, for example, higher vapor heat capacity, higher liquid heat capacity, lower latent heat-to-heat capacity ratio, higher critical temperature, and higher thermal stability as well as acceptable environmental, flammability, and toxicological properties would represent improvements over fluids such as HFC-245fa and HCFC-123.

The art is continually seeking new fluorocarbon based working fluids which offer alternatives for refrigeration, heat pump, foam blowing agent and energy generation applications. Currently, of particular interest, are fluorocarbon—based compounds which are considered to be environmentally safe substitutes for fully and partially halogenated fluorocarbons (CFCs and HCFCs) such as trichlorofluoromethane (CFC-11), 1,1,-dichloro-1-fluoroethane (HCFC-141b) and 1,1-dichloro-2.2-trifluoroethane (HCFC-123) which are regulated in connection with the need to conserve the earth's protective ozone layer. Similarly, fluids that have a low global warming potential (affecting global warming via direct emissions) or low life cycle climate change potential (LCCP), a system view of global warming impact, are desireable. In the latter case, organic Rankine cycle improves the LCCP of many fossil fuel driven power generation systems. With improved overall thermal efficiency, these systems that incorporate organic Rankine cycle can gain additional work or electric power output to meet growing demand without consuming additional fossil fuel and without generating additional carbon dioxide emissions. For a fixed electric power demand, a smaller primary generating system with the organic Rankine cycle system incorporated can be used. Here, too, the fossil fuel consumed and subsequent carbon dioxide emissions will be less compared to a primary system sized to supply the same fixed electric power demand. The substitute materials should also possess chemical stability, thermal stability, low toxicity, non-flammability, and efficiency in-use, while at the same time not posing a risk to the planet's atmosphere. Furthermore, the ideal CFC/HCFC refrigerant substitute should not require major engineering changes to conventional technology currently used with CFC/HCFC materials. It should also be compatible with commonly used and/or available materials of construction.

### SUMMARY OF THE INVENTION

It has been discovered that polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof, and especially methyl(perfluoropropyl)ether, methyl(perfluorobutyl)ether and perfluoroethyl perfluoroisopropyl ketone and mixtures thereof, meet the requirement for not adversely affect atmospheric chemistry and would be a negligible contributor to ozone depletion and to green-house global warming in comparison to the fully halogenated hydrocarbons and are suitable for use as working fluids for use in thermal energy conversion systems. Thus, in a method for converting thermal energy to mechanical energy, particularly using an organic Rankine cycle system, polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl (trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof, and especially methyl(perfluoropropyl)ether, methylperfluorobutyl)ether and perfluoroethyl perfluoroisopropyl ketone and mixtures thereof, have been found to be particularly useful as a working fluid in such energy conversion systems.

One of the properties of HFC propanes, shared by the polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof, and especially methyl(perfluoropropyl)ether, methylperfluorobutyl)ether and perfluoroethyl perfluoroisopropyl ketone and mixtures thereof, working fluids of this invention, that makes their use in heat to mechanical conversions advantageous is the entropy/temperature relationship at saturated vapor conditions. Heat energy can be converted to mechanical energy in a Rankine cycle in a process known as isentropic expansion. For example, as the gas at a higher temperature and pressure is expanded through a turbine to a region of lower pressure, it does work upon the turbine, exiting the turbine at a lower pressure and temperature. The difference in the enthalpies of the gas between the two points is equal to the amount of work that the gas does on the turbine. If the higher temperature, higher pressure gas has a decrease in its entropy as the temperature pressure is lowered, the gas will not condense in an isentropic expansion; in other words, it will not partially liquify as it drops in temperature and pressure across the turbine. Such condensation can cause unwanted wear and tear on the mechanical device (turbine, in this case), and can only be overcome by superheating the vapor prior to its entering the turbine. For small molecular species such as water, ammonia and dichlorodifluoromethane, superheating of the vapor is required to prevent significant condensation during an isentropic expansion. However, for larger molecules such as HCFC-123, HFC-245fa, and the compounds of this invention, the entropy increases as the temperature is raised (in a saturated vapor), and condensation will not occur in an isentropic expansion.

One embodiment of the invention compises a process for converting thermal energy to mechanical energy in a Rankine cycle in which a cycle is repeated comprising the steps of vaporizing a working fluid with a hot heat source, expanding the resulting vapor and and then cooling with a cold heat source to condense the vapor, and compressing the working fluid, wherein the working fluid is selected from polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof, and especially methyl(perfluoropropyl)ether, methyl(perfluorobutyl)ether, perfluoroethyl perfluoroisopropyl ketone and mixtures thereof.

Another embodiment of the invention comprises a process for converting thermal energy to mechanical energy which comprises heating a working fluid to a temperature sufficient to vaporize the working fluid and form a pressurized vapor of the working fluid and then causing the pressurized vapor of the working fluid to perform mechnical work, wherein the working fluid comprises a working fluid selected from the group consisting of polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof, and especially methyl(perfluoropropyl)ether, methyl(perfluorobutyl)ether and perfluoroethyl perfluoroisopropyl ketone and mixtures thereof.

A further embodiment of the invention comprises a binary power cycle comprising a primary power cycle and a secondary power cycle, wherein high temperature water vapor is the primary working fluid in the primary power cycle, and a second working fluid is employed in the scondary power cycle to convert hthermal enery to mechanical energy and is heated to form a pressurized vapor of the second working fluid and the pressurized vapor of the second working fluid is caused to perform mechanical work, wherein the working fluid comprises a working fluid selected from polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof, and especially methyl(perfluoropropyl)ether, methyl(perfluorobutyl)ether and perfluoroethyl perfluoroisopropyl ketone and mixtures thereof.

### DETAILED DESCRIPTION OF THE INVENTION AND PREFERRED EMBODIMENTS

It has been discovered that polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof, and especially methyl(perfluoropropyl)ether, methyl(perfluorobutyl)ether and perfluoroethyl perfluoroisopropyl ketone and mixtures thereof, meet the requirement for not adversely affect atmospheric chemistry and would be a negligible contributor to ozone depletion and to green-house global warming in comparison to the fully halogenated hydrocarbons and are suitable for use as working fluids for use in thermal energy conversion systems. Thus, in a method for converting thermal energy to mechanical energy, particularly using an organic Rankine cycle system, polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof, and especially methyl(perfluoropropyl)ether, methyl perfluorobutyl)ether and perfluoroethyl perfluoroisopropyl ketone and mixtures thereof, have been found to be particularly useful as a working fluid in such energy conversion systems. Mathematical models have substantiated that such compounds, and especially methyl(perfluoropropyl)ether, methylperfluorobutyl)ether and perfluoroethyl perfluoroisopropyl ketone and mixtures thereof will not adversely affect atmospheric chemistry, being a negligible contributor to ozone depletion and to green-house global warming in comparison to the fully halogenated hydrocarbons.

In accordance with the invention, we have found that the fluids of polyfluorinated ethers and polyfluorinated ketones and mixtures thereof, preferably polyfluorinated ethers such as methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), and polyfluorinated ketones such as methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof, and especially methyl(perfluoropropyl)ether, methyl(perfluorobutyl)ether and perfluoroethyl perfluoroisopropyl ketone and mixtures thereof, are useful as energy conversion fluids. A list of some of their properties of the especially preferred fluids of this invention is shown in Table 1 in comparison to the properties of HFC-134fa and HCFC-123. Interestingly, the work output that can be derived in an organic Rankine cycle is not only higher for the fluids having a higher critical temperature when the boiler temperature is allowed to approach the critical temperature (moving toward higher cycle efficiency), but it is also unexpectedly higher for those same higher critical temperature fluids when the boiler temperature is constrained so as to be measurably lower than the critical temperature as when a lower grade heat source is available.

As such, the present invention meets the need in the art for a working fluid which has a low ozone depletion potential and is a negligible contributor to green-house global warming compared with fully halogenated CFC and partially halogenated HCFC materials, is effectively nonflammable, of low toxicity and is chemically/thermally stable in conditions where it is likely to be employed. These materials have the proper boiling points and thermodynamic characteristics that would be usable in thermal energy conversion to mechanical shaft power and electric power generation; they could take advantage of some of the latent heat contained in low pressure steam that is presently not well utilized. The above listed materials may be employed to extract additional mechanical energy from low grade thermal energy sources such as industrial waste heat, solar energy, geothermal hot water, low-pressure geothermal steam (primary or secondary arrangements) or distributed power generation equipment utilizing fuel cells or prime movers such as turbines, microturbines, or internal combustion engines. Low-pressure steam can also be accessed in a process known as a binary Rankine cycle. Large quantities of low pressure steam can be found in numerous locations, such as in fossil fuel powered electrical generating power plants. Binary cycle processes using these working fluids would prove especially useful where a ready supply of a naturally occuring low temperature “reservoir”, such as a large body of cold water, is available. The particular fluid could be tailored to suit the power plant coolant quality (its temperature), maximizing the efficiency of the binary cycle.

TABLE 1
Properties Comparison

| | | Heat | | | Flame |
|---|---|---|---|---|---|
| | | Capacity | | | Limits, |
| | | (liquid, | Boiling | Permissible | Vol. % |
| | | 25° C.), | Point, | Exposure | At 1 |
| | ODP/GWP* | J/g-mol K | ° C. | Level, ppm | atmosphere |
| Methyl | 0/370 | 260.0 | 34.0 | 75 | None |
| (perfluoropropyl) ether | | | | | |
| Perfluoroethyl | 0/1 | 348.5 | 49.2 | None | None |
| perfluoroisopropyl | | | | assigned | |
| ketone | | | | | |
| Methyl (perfluorobutyl) | 0/320 | 293.8 | 61.1 | 750 | None |
| ether | | | | | |
| HFC-245fa | 0/950 | 185.5 | 14.9 | 300 | None |
| HCFC-123 | 0.02/110 | 160.3 | 27.8 | 30 | None |

*Ozone Depletion Potential/Global Warming Potential

The present invention is more fully illustrated by the following non-limiting examples.

### EXAMPLE 1

Work Output at Optimum Cycle Efficiency for a Sub-Critical Organic Rankine Cycle Process

In organic Rankine cycle thermal to mechanical energy applications, an increased cycle efficiency can be shown to be possible if the working fluid is properly matched to the source and sink temperature. In this example, the sink temperature is considered to be fixed in order to highlight the interrelationship between critical temperature and efficiency and work output. With regard to the source temperature, a cycle irreversibility that decreases efficiency is the temperature difference between the evaporating condition and the source temperature. Minimizing this “gap” in temperature results in an improvement in the cycle efficiency. Additionally, the higher the working fluid critical temperature (increased ability to access higher thermal energy sources) the more work output that is possible. Hence, in Table 2, it can be seen that the compounds of the invention, which have higher critical temperatures than HFC-245fa, have their respective highest work output when the cycle efficiency is at a maximum for each fluid. At maximum cycle efficiency, each of the fluids of the invention have higher work output than HFC-245fa. In the table, the cycle efficiencies of methyl pefluorobutyl ether and HFC-245fa are comparable when each is operating with a boiler temperature 5° F. (2.8° C.) below the corresponding critical temperature. Even when the cycle efficiencies are comparable, the work output of the methyl perfluorobutyl ether exceeds that of HFC-245fa. This example demonstrates the usefulness of the fluids of the invention in organic Rankine cycle applications. Conditions for the example are chosen such that the expansion process starts at saturated vapor condition or just slightly superheated. Because the fluids of the example have more superheat at the exit of the expander than at the entrance of the expander, more heat will be rejected without being used if entrance superheats are increased (unless some form of recuperation is used). The condensing condition was selected to be that of a cold water condensing source such as a deep lake which again is desireable in that it contributes to an increased cycle efficiency and work output as compared to a higher condensing temperature of, for example, air-cooled condensation. To arrive at the net work output, the absolute value of the enthalpy gain i n the p ump due to the increase in pressure in going from the condensing condition back to the boiler condition is subtracted from the absolute value of the difference in enthalpy values at the expander entrance (boiler exit) and expander exit. Dividing “net work output” by “heat input” to the boiler provides the cycle efficiency. In order to model thermodynamics of the compounds of the invention, an equation of state must be selected. The corresponding-states principle is probably the most effective basis for thermophysical property estimation. Lee and Kesler (Lee, B. I. And Kesler, M. G.; AlChE J.; 21; 510 (1975)) developed an extended corresponding states method that utilizes two reference fluids in order to predict thermodynamic properties. The two reference fluid orginally chosen by Lee and Kesler were a “simple fluid” and n-octane. If thermodynamic properties of a particular family of compounds (e.g., flourinated compounds) is desired the accuracy of the Lee and Kesler method can be improved by substituing the n-octane reference fluid with a reference fluid from the family of interest. Sörner and Strom (Sörner, M. and Ström; Can. J. Chem. Eng.; 73; 854 (1995)) reported the constants needed to substitute HFC-134a (1,1,1,2-tetrafluorotheane) as the reference fluid. If HFC-134a replaces n-octane as the reference fluid the resulting extended corresponding states method can now be used to accuratly model the thermodynamic properties of flourinated compounds. Now that an equation of state has been selected the properties of a Rankine cycle are calculated following the procedure described by Smith, Van Ness and Abbott (Smith, J. M., Van Ness, H. C. and Abbott, M. M.; Introduction to Chemical Engineering Thermodynamics; The McGraw-Hill Companies, Inc; New York (1996)). These properties were used to calculate “net work out” for the expansion step and cycle efficiency. Required inputs for this estimation include critical temperature, critical pressure, acentric factor and ideal gas heat capacity. The pump and compressor in the Rankine cycle were assumed to operate isentropically. Values of the critical temperature and critical pressure were taken from the literature. In the case of methyl pefluorobutyl ether, critical pressure was estimated using Joback's method (Joback, K. G. and Reid R. C.; Chem. Eng. Comm.; 57; 233 (1987)). For all the fluids except HFC-245fa , the ideal gas heat capacity was calculated using commercially available CS Chem 3D Pro software from Cambridge Soft. In the case of HFC-245fa, the ideal gas heat capacity was obtained using NIST Refprop 6.01 Software available from the National Institute of Standards and Technology. Acentric factors for the fluorinated ethers and fluorinated ketone were calculated using Reidels method (Riedel, L; Chem. Ing. Tech.; 26; 679 (1954)).

TABLE 2
Increased work output and efficiency - boiler near critical temperature

| | HFC- | HFC- | | | PFE- | PFE- | | |
|---|---|---|---|---|---|---|---|---|
| | 245fa₍₁₎ | 245fa | MPFPE | mPFPE | PFIPK₍₂₎ | PFIPK | MPFBE₍₃₎ | mPFBE |
| Condenser | 53.10 | 53.10 | 53.10 | 53.10 | 53.10 | 53.10 | 53.10 | 53.10 |
| Temp. ° F. | (11.7) | (11.7 | (11.7 | (11.7 | (11.7) | (11.7) | (11.7) | (11.7) |
| (° C.) | | | | | | | | |
| Condenser | 13.22 | 13.22 | 6.28 | 6.28 | 4.12 | 4.12 | 2.00 | 2.00 |
| Pressure psia | | | | | | | | |
| Boiler | 250 | 300.00 | 300.00 | 324.00 | 300.00 | 330.66 | 300.00 | 378.00 |
| Temperature | (121) | (149) | (149) | (162.2) | (149) | (165.9) | (149) | (192.2) |
| ° F. (° C.) | | | | | | | | |
| Boiler | 284.2 | 458.4 | 269.3 | 334.4 | 186.5 | 257.6 | 126.6 | 276.7 |
| Pressure psia | | | | | | | | |
| ° F. Super | 0.0 | 1.3 | 0.0 | 0.7 | 0.0 | 0.0 | 0.00 | 0.30 |
| heated | (0.0) | (0.72) | (0.0) | (0.39) | (0.0) | (0.0) | (0.0) | (0.16) |
| (° C.) | | | | | | | | |
| Critical | 309.29 | 309.29 | 329.00 | 329.00 | 335.66 | 335.66 | 383.0 | 383.0 |
| Temperature | (151.27) | (151.27) | (165) | (165) | (168.7) | (168.7) | (195) | (195) |
| ° F. (° C.) | | | | | | | | |
| Critical | 527.90 | 527.90 | 359.70 | 359.70 | 270.50 | 270.50 | 292.2 | 292.2 |
| Pressure psia | | | | | | | | |
| Net Work | 3117.80 | 3487.6 | 4055.3 | 4214.4 | 4658.8 | 4870.9 | 5103.29 | 5976.2 |
| BTU/lbmol | | | | | | | | |
| Thermal | 0.207 | 0.227 | 0.220 | 0.226 | 0.197 | 0.203 | 0.212 | 0.229 |
| Efficiency | | | | | | | | |

mPFPE—methyl perfluoropropyl ether
mPFBE—methyl perflurorbutyl ether
PFE-PFIPK-perfluoroethyl perfluoroisopropyl ketone
₍₁₎HFC-245fa critical temperature, critical pressure, and ideal gas heat capacity from NIST Refprop 6.01 Software, environmental and other physical properties from Honeywell Genetron ® 245fa Applications Development Guide.
₍₂₎Perfluoroethyl perfluoroisopropyl ketone boiling point, liquid heat capacity, critical temperature, critical pressure, GWP and ODP from 3M Novec ™ 1230 Fire Protection Fluid data sheet.
₍₃₎Methy perfluoropropyl ether and methyl perfluorobutyl ether critical temperature, critical pressure, heat capacity, boiling point, ODP, GWP, and permissible (recommended) exposure level from 3M Novec ™ Engineered Fluid HFE-7000 product information sheet and HFE-7100 for Heat Transfer Data Sheet, respectively.

### EXAMPLE 2

Work Output at Cycle Efficiency Away from Optimum, Organic Rankine Cycle Process

One way to assess the the relative “goodness” of organic Rankine cycle working fluids is to compare theoretical cycle efficiency. However, many organic Rankine cycle systems utilize waste heat as the driver, hence cycle efficiency is typically not as important as the net work derived (work extracted during expansion less work of the pump). Example 1 compares the net work derived for HFC-245fa, methyl perfluoropropyl ether(1-methoxyheptafluoropropane), methyl perfluorobutyl ether(1-methoxynonafluorobutane) and perfluoroethyl perfluoroisopropyl ketone(dodecafluoro-2-methylpentan-3-one) with a particular focus on work output at maximum cycle efficiency. In Example 1 it was shown that it is desired to select an evaporating temperature that is near the working fluid critical temperature in order to maximize cycle efficiency and the work extracted. Thermodynamic properties used to determine the values in Table 3 were estimated as described in Example 1. In Table 3, the net work output was higher, i.e., more desirable, for the fluorinated ketone and fluorinated ethers of the invention as compared to HFC-245fa even though the boiler temperature and cycle efficiency was below the optimum for the fluids of the invention (lower boiler temperature/lower efficiency cases in Example 1) are compared to the case where HFC-245fa work output and cycle efficiency are a maximum. Demonstrating higher work output even though the cycle was not operating at optimum conditions for the given working fluid highlights an unexpected benefit of utilizing the working fluids of the invention.

TABLE 3
Net WorkOutput away from optimum cycle efficiency

| | | | Net Work |
|---|---|---|---|
| | Evaporating | Condensing | Out, |
| | Temperature | Temperature | Btu/lb- |
| Compound | ° F. (° C.) | ° F. (° C.) | mole |
| HFC-245fa | 300 (149) | 53 (12) | 3487.6 |
| Perfluoroethyl | 300 (149) | 53 (12) | 4658.8 |
| perfluoroisopropyl ketone | | | |
| Methyl perfluoropropyl ether | 300 (149) | 53 (12) | 4055.3 |
| Methyl perfluorobutyl ether | 300 (149) | 53 (12) | 5103.3 |

It is also possible to compare the relative performance of working fluids by using an empirical formula which relates to field observations. In Example 3, Equation 1 approximates the electric power that can be generated for a given source temperature in a geothermal applicaton. This empirical relationship takes into account typical heat exchanger approach temperature, source temperature, and typical sink temperature. It can be argued that rather than inputting source temperature, substitution of critical temperature of the working fluid results in a relative comparison of working fluids taking into account differences in the magnitude of the irreversibility related to the difference in evaporating temperature (linked to the limiting factor of critical temperature for sub-critical cycles) and source temperature. Even though this substitution may underestimate the power output, it holds that the higher the resultant electric power output, the more effective the working fluid.

### EXAMPLE 3

Minimizing the temperature difference betweeen the cycle evaporating temperature and the source temperature minimizes an irreversibility in the cycle and thus improves the efficiency. Thus, for two otherwise similar working fluids, the one with the higher critical temperature will allow for operation of the evaporator in the organic Rankine cycle at temperatures closer to the source temperature and therefore will demonstrate a higher cycle efficiency. An empirical relationship exists which can be modified so as to reflect these efficiency differences. Equation 1 can be used to reasonably estimate the electric power that can be derived using organic Rankine cycle to convert thermal energy from a flowing fluid source. Typically, this equation would relate to geothermal applications.
NEP=[(0.18T−10)ATP]/278
where: T is the inlet temperature of the flowing source fluid (° C.),
NEP is the net electric power (kW), and
ATP is the available thermal power (kW).

For this example, it is assumed that thermal power is only accessed significantly at a secondary fluid temperature range from the working fluid critical temperature (Tc) down to 50° C. Since this is a relative comparison, a secondary fluid flow rate for sensible heating of 1 kg/sec was chosen. Water is used as the secondary fluid. The available thermal power is calculated from the equation mCp(Tc-50) where mass flow rate (m) is 1 kg/sec and the specific heat of water (Cp) is 4.186 kJ/kg. The available thermal power is the heat available from the flowing source fluid and is typically determined using a temperature 10° C. above the bottom cycle temperature. In this case, the lower limit of 50° C. was chosen. Instead of using the source temperature for T, the critical temperature (Tc) of each fluid is used since it accounts for differences in the approach of the working fluid relative to the source temperature. Hence, the relative ranking of working fluids based on the calculated electrical output of this example found in Table 4 below shows that methyl(perfluoropropyl)ether, methyl(pefluorobutyl)ether and perfluoroethyl perfluoroisopropyl ketone represent improvements over HCFC-123 and HFC-245fa. The thermal limitation on HCFC-123 is necessary to avoid formation of toxic HCFC-133a via thermal decomposition. Application of Equation 1 to HCFC-123 at its critical temperature is not practical.

TABLE 4
Relative Ranking of Working Fluids Based on Calculated Net
Electric Power Output (NEP)

| | Critical | | |
|---|---|---|---|
| | Temperature, Tc | | |
| COMPOUND | (° C.) | ATP | NEP |
| Methyl (perfluoropropyl) | 165.0 | 481.4 | 34.1 |
| ether | | | |
| Perfluoroethyl | 168.7 | 496.9 | 36.4 |
| perfluoroisopropyl ketone | | | |
| Methyl (perfluorobutyl) | 195.3 | 607.0 | 54.8 |
| ether | | | |
| HCFC-123 | 183.6 | N/A | N/A |
| | | (not applicable) | |
| HCFC-123 with thermal | 121.0 | 297.2 | 12.6 |
| limitation | (maximium | | |
| | temperature) | | |
| HFC-245fa | 151.1 | 423.2 | 26.2 |

The compounds of the invention possess several features that are beneficial to 1 thermal energy conversion to produce shaft power and/or subsequently electric power. Some of these include the low viscosity of these fluids (lowers pumping losses), the large molecular weight of the compounds and corresponding high densities of the vapors compared to similar pressured steam, the low latent heat of vaporization and good heat transfer properties. Turbines that are designed to use these materials would have a greater energy output per unit volume than a steam turbine at the same pressures. This could become especially important, since low-pressure turbines in large energy plants can be large in size as well as cost.

While the invention has been described herein with reference to the specific embodiments thereof, it will be appreciated that changes, modification and variations can be made without departing from the spirit and scope of the inventive concept disclosed herein. Accordingly, it is intended to embrace all such changes, modification and variations that fall with the spirit and scope of the appended claims.

1. A process for converting thermal energy to mechanical energy in a rankine cycle in which a cycle is repeated comprising the steps of vaporizing a working fluid with a heat source, expanding the resulting vapor and then cooling with a cold heat source to condense the vapor, and pressurizing the working fluid, wherein the working fluid comprises a working fluid selected from the group consisting of polyfluorinated ethers, polyfluorinated ketones and mixtures thereof, and wherein the polyfluorinated ethers are not fully halogenated.

2. A process according to claim 1 wherein the working fluid is selected from the group consisting of methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof.

3. A process according to claim 1 wherein the working fluid is selected from the group consisting of methyl(perfluoropropyl)ether, methyl(perfluorobutyl)ether, perfluoroethyl perfluoroisopropyl ketone and mixtures thereof.

4. A process according to claim 3 wherein the working fluid comprises methyl(perfluoropropyl)ether.

5. A process according to claim 3 wherein the working fluid comprises methyl(perfluorobutyl)ether.

6. A process according to claim 3 wherein the working fluid comprises perfluoroethyl perfluoroisopropyl ketone.

7. A process for converting thermal energy to mechanical energy which comprises heating a working fluid to a temperature sufficient to vaporize the working fluid and form a pressurized vapor of the working fluid and then causing the pressurized vapor of the working fluid to perform mechanical work, wherein the working fluid comprises a working fluid selected from the group consisting of polyfluorinated ethers, polyfluorinated ketones and mixtures thereof, and wherein the polyfluorinated ethers are not fully halogenated.

8. A process according to claim 7 wherein the working fluid is selected from the group consisting of methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof.

9. A process according to claim 7 wherein the working fluid is selected from the group consisting of methyl(perfluoropropyl)ether, methyl(perfluorobutyl)ether, perfluoroethyl perfluoroisopropyl ketone and mixtures thereof.

10. A process according to claim 9 wherein the working fluid comprises methyl(perfluoropropyl)ether.

11. A process according to claim 9 wherein the working fluid comprises methyl(perfluorobutyl)ether.

12. A process according to claim 9 wherein the working fluid comprises perfluoroethyl perfluoroisopropyl ketone.

13. A process according to claim 7 wherein the pressurized vapor of the working fluid is subsequently cooled below its boiling point and then recycled by again heating the working fluid to again form a pressurized vapor of the working fluid which is then caused to perform additional mechanical work.

14. A binary power cycle comprising a primary power cycle and a secondary power cycle, wherein high temperature water vapor is the primary working fluid in the primary power cycle, and a second working fluid is employed in the scondary power cycle to convert thermal energy to mechanical energy and is heated to form a pressurized vapor of the second working fluid and the pressurized vapor of the second working fluid is caused to perform mechanical work, wherein the working fluid comprises a working fluid selected from the group consisting of polyfluorinated ethers, polyfluorinated ketones and mixtures thereof, and wherein the polyfluorinated ethers are not fully halogenated.

15. A binary power cycle according to claim 14 wherein the working fluid comprises a working fluid selected from the group consisting of methyl(trifluoroethyl)ether (CH₃OCH₂CF₃), methyl(heptafluoropropyl)ether (CH₃OCF₂CHFCF₃), di(trifluoroethyl)ether (CF₃CH₂OCH₂CF₃), methyl(hexafluoropropyl)ether (CH₃OCF₂CF₂CHF₂), methyl(pentafluoropropyl)ether (CH₃OCH₂CF₂CF₃), methyl(perfluorobutyl)ether (C₄F₉OCH₃), ethyl(perfluorobutyl)ether (C₄F₉OC₂H₅), methyl(perfluoromethyl)ketone (CF₃COCH₃), perfluoromethyl(trifluoroethyl)ketone (CF₃CH₂COCF₃), methyl(perfluooroethyl)ketone (C₂F₅COCH₃), methyl(perfluoropropyl)ketone (F₃CF₂CF₂COCH₃), perfluoroethyl(perfluoropropyl)ketone (CF₃CF₂CF₂COC₂F₅), methyl(octafluorobutyl)ketone (C₂F₅CFHCF₂COCH₃), di(perfluoropropyl)ketone (CF₃CF₂CF₂COCF₂CF₂CF₃), and mixtures thereof.

16. A binary power cycle according to claim 14 wherein the working fluid is selected from the group consisting of methyl(perfluoropropyl)ether, methyl(perfluorobutyl)ether, perfluoroethyl perfluoroisopropyl ketone and mixtures thereof.

17. A binary power cycle according to claim 15 wherein the working fluid comprises methyl(perfluoropropyl)ether.

18. A binary power cycle according to claim 15 wherein the working fluid comprises methyl(perfluorobutyl)ether.

19. A binary power cycle according to claim 15 wherein the working fluid comprises perfluoroethyl perfluoroisopropyl ketone.
